(19) United States
(12) Patent Application Publication
(10) Pub. No.: US 2015/0226824 A1
(43) Pub. Date: Aug. 13, 2015

(54) METHOD AND MAGNETIC RESONANCE SYSTEM FOR GENERATING MR IMAGES

(71) Applicant: Siemens Aktiengesellschaft, Muenchen (DE)

(72) Inventors: Robert Grimm, Nuernberg (DE); Marcel Dominik Nickel, Herzogenaurach (DE)

(73) Assignee: Siemens Aktiengesellschaft, Muenchen (DE)

(21) Appl. No.: 14/620,611

(22) Filed: Feb. 12, 2015

(30) Foreign Application Priority Data
Feb. 12, 2014 (DE) 102014202513.3

(51) Int. Cl. G01R 33/58; A61M 5/00; G01R 33/36; G01R 33/48; G01R 33/30; A61B 5/055; G01R 33/561

(52) U.S. Cl. CPC G01R 33/583; A61B 5/055; A61M 5/007; G01R 33/561; G01R 33/5616; G01R 33/4826; G01R 33/4822; G01R 33/30; G01R 33/3621; A61B 2576/00

(57) ABSTRACT

In a method and a magnetic resonance (MR) system for generating MR images, MR data of a predetermined volume segment within an examination subject are acquired using the same measurement configuration of the MR system. A number of MR images are reconstructed from the MR data. Each of the MR images is assigned to a respective time point at which the MR image represents at least a part of the volume segment. A spatial resolution during the acquisition of the MR data is maintained constant because of the aforementioned same measurement configuration. The temporal distance between each two time points succeeding one another in time is not constant.

### BACKGROUND OF THE INVENTION

1. Field of the Invention

The present invention relates to a method for generating MR images or reconstructing the MR images from MR data, as well as to a magnetic resonance system for implementing such a method.

2. Description of the Prior Art

Known measurement and acquisition strategies according to the prior art enable MR data to be acquired with a high temporal resolution, as a result of which MR images having a high temporal resolution can be reconstructed. If the total time interval in which said MR data is acquired must be selected so as to be sufficiently long in order, for example, to track the diffusion of a contrast agent, the reconstruction of the MR images requires a correspondingly long period of time, which is determined by the computing time for the reconstruction. The disadvantage of such a long period of time, which, depending on the total time interval and the temporal resolution, may very well lie in the hours range, is firstly that computer resources are occupied for a correspondingly long time, and that the assessment of the MR images has to be deferred for a correspondingly long time.

### SUMMARY OF THE INVENTION

An object of the present invention is to accelerate the reconstruction of the MR images, but without the cost of significant penalties in terms of the quality of the reconstructed MR images.

This object is achieved by a method for generating MR images is provided in accordance with the invention, wherein MR data of a predetermined volume segment within an examination subject are acquired with a magnetic resonance system, the MR data being acquired using the same measurement configuration. Multiple MR images are reconstructed from the acquired MR data. Each of the MR images is assigned to an individual time point at which the reconstructed MR image represents a section of the volume segment corresponding to the MR image.

The measurement configuration keeps the spatial resolution constant while acquiring the MR data. For example, in a slice-by-slice acquisition of the MR data, each slice is acquired at the same resolution. According to the invention the temporal distance (separation) between each two successive time points is not constant, but varies. In other words, a first temporal distance between a first of the time points and a second of the time points, which follows directly in time after the first time point, is different from a second temporal distance between the second time point and a third of the time points, which follows directly in time after the second time point.

Because the temporal distance between two MR images succeeding one another in time varies according to the invention, the temporal distance may be chosen, for example, so as to be relatively small in clinically interesting states (for example during the takeup of a contrast agent) and to be relatively large in clinically uninteresting states. Consequently, more MR images per unit time are reconstructed in clinically interesting states, as a result of which the temporal distance between succeeding MR images is relatively small. Because the reconstructed MR images are present with a high temporal resolution only in clinically interesting states and in other states are present with a correspondingly low temporal resolution, the total number of MR images that are to be reconstructed is lower in comparison with the prior art, as a result of which the time required for reconstructing the MR images is advantageously shortened. In other words, the reconstruction time can be significantly reduced compared to the prior art, without the necessity of accepting significant penalties in terms of quality, since the temporal resolution of the reconstructed MR images in clinically interesting states can be just as high as in the prior art. In summary, the present invention enables the MR images to be reconstructed non-equidistantly with respect to time.

The MR data or raw data are acquired using a single (the same) measurement configuration. This does not preclude the acquisition of the MR data being able to be performed with interruptions or with multiple separate measurements, all of which employ the same measurement configuration, although acquiring the MR data on the basis of only one measurement is preferred. As used herein, the measurement configuration means the calibration of the magnetic resonance system, which in turn includes setting the transmitting power of the RF antenna(s), setting the reception sensitivity of the RF antenna(s), and/or setting the excitation frequency, which includes selecting the sequence protocol. Stated differently, the acquisition of the MR data generally takes place using the same sequence protocol.

According to an inventive embodiment, the temporal distance between each two time points succeeding one another in time, i.e. between two temporally sequential MR images that are to be reconstructed, is determined dependent on information that describes a change occurring within the volume segment.

The change that occurs can concern, for example, the diffusion of a contrast agent in the volume segment. It is also possible for the change that occurs to involve the heartbeat or the respiration of the examination subject when, for example, MR images are to be selectively generated during a specific heartbeat phase or breathing position.

The aforementioned information can be, for example, the time point at which a contrast agent is injected into the examination subject. According to another inventive variant, the time curve of a contrast agent concentration after a trial injection of a contrast agent into the examination subject is provided as the information.

According to an inventive variant, the information that describes the occurring change is ascertained on the basis of the acquired MR data, without the need for MR images to have been reconstructed beforehand from the acquired MR data. Such a variant corresponds to a technique known as self-gating, since the temporal resolution of the MR images required in each case is determined automatically.

When, for example, a contrast agent diffuses in the volume segment, the contrast within the volume segment increases, thereby increasing the transverse magnetization overall within the volume segment, as a result of which finally the amplitude of the raw data values (i.e. of the acquired MR data) is increased on an average. In other words, the absolute amount of the raw data values increases, the more contrast agent has diffused in the volume segment. The concentration of the contrast agent in the volume segment thus can be deduced, for example, by a simple averaging of the acquired MR data.

In this case the information can also be ascertained only from the acquired MR data lying in the center of a k-space slice (k, =0) or in the center of the k-space.

Since the k-space points in the center of a slice and consequently also in the center of k-space provide the essential information relating to the contrast of an MR image, it is advantageously sufficient if, for example during the acquisition of the MR data along radial spokes in k-space that intersect the z-axis, to determine only the value of the k-space point that corresponds to the point of intersection of the radial spoke with the z-axis, or an average value of a specific number (e.g. 3) of k-space points that lie on the spoke in the vicinity of the center of k-space. In this case, the z-axis extends in the direction of the basic magnetic field and effectively corresponds to the central axis of the volume that is excited by the magnetic field of the magnetic resonance system.

The information can correspond to a first time point at which an increase in a contrast agent concentration in the volume segment is deduced as a function of the acquired MR data, and a second time point at which an end to this increase in the contrast agent concentration in the volume segment is deduced as a function of the acquired MR data.

If the distribution of a contrast agent in the body of an examination subject is tracked by evaluation of MR images, the time range of most interest is that time range in which the concentration of the contrast agent increases in the observed volume segment of the examination subject. The first and the second time points that are derived from the acquired MR data are therefore particularly important.

In particular, the temporal distance between the MR images to be reconstructed between the first time point and the second time point is kept as small as possible so that the temporal resolution in the time range between the first and the second time points is as high as possible. By comparison, the temporal resolution at a time prior to the first time point or at a time following the second time point can turn out to be smaller.

Since the time range between the first and the second time points is of special interest, the temporal resolution of the MR images to be reconstructed in this time range is set as high as possible so that as many MR images as possible are reconstructed or present in this time range.

According to another inventive embodiment, the information describing a change occurring within the volume segment is derived from the reconstructed MR images.

In this embodiment, the corresponding information at a specific time point can be ascertained, for example, as a function of MR images reconstructed on the basis of MR data acquired prior to the specific time point. The temporal resolution of those MR images reconstructed on the basis of MR data acquired after this time point can then be set as a function of the information ascertained at the specific time point.

The information can be the specification of a time point at which the contrast agent is injected into the examination subject.

In this embodiment it is not necessary to evaluate the acquired MR data or the previously reconstructed MR images in order to ascertain the information, because the information corresponds, for example, to the time point specified by the treating physician, at which the contrast agent is or was injected into the examination subject.

According to an inventive embodiment, one of the MR images, some of the MR images, or all of the MR images is or are reconstructed in each case from that MR data acquired in a first predetermined time period prior to, and a second predetermined time period after, the time point that is assigned to the respective MR image. In this case the first predetermined time period can be equal to the second predetermined time period so that the volume of MR data acquired prior to the time point corresponds to the volume of MR data acquired after the time point.

In this embodiment, in particular for reconstructing one MR image, only MR data are used that were acquired for a time interval in which the time point assigned to the MR image lies. Since the time intervals are not selected as equidistant, the temporal distance between the reconstructed MR images or the temporal resolution of the reconstructed MR images will also be varied accordingly.

According to the invention, all of the acquired MR data may be used for the reconstruction of the MR images.

In this embodiment there is no acquired MR data that is not taken into account during the reconstruction.

It is also possible not to use certain acquired MR data for the reconstruction when, for example, the temporal distance between two successive MR images that are to be reconstructed is too great. For example, if the temporal distance between a first time point assigned to a first MR image that is to be reconstructed and a second time point assigned to a second MR image that is to be reconstructed and directly follows the first MR image in time is greater than a predetermined time threshold value, a part of the MR data acquired between the first time point and the second time point may not be used for the reconstruction of an MR image.

In similar fashion it is also possible not to acquire certain MR data at all when, for example, the temporal distance between two successive MR images to be reconstructed is too great. For example, if the temporal distance between a first time point assigned to a first MR image that is to be reconstructed and a second time point assigned to a second MR image that is to be reconstructed and directly follows the first MR image in time is greater than the predetermined time threshold value, a measurement pause in which no MR data is acquired can be inserted between the first and the second time point.

If the temporal distance between the first and the second time points is greater than the predetermined time threshold value, more MR data may be acquired for an MR image to be reconstructed than is necessary for the reconstruction. In order not to prolong the reconstruction due to the quasi-superfluous MR data, this MR data may actually be acquired, but not be taken into account for the reconstruction, or MR data may not be acquired at all in the first place.

Generally in the case of the present invention, more MR data are available for the reconstruction of an MR image and can be used, the greater the temporal distance is between the first and the second time points. In other words, more MR data is available per MR image, the lower or, as the case may be, the poorer, the temporal resolution is of the MR images that are to be reconstructed.

The temporal resolution at which the MR data is acquired may be constant.

Depending on the sequence protocol employed, an excitation step is usually performed, this being followed by a readout step in which the MR data is acquired. The repetition time TR (Time to Repetition) is defined as the time period from the start of an excitation step to the start of the next-following excitation step. Usually said time-to-repetition TR is constant, meaning that a constant amount of MR data is acquired per unit time.

According to the invention, it is also possible for the temporal resolution not to be constant because, for example, no MR data is acquired at certain times.

In the previously described embodiments, the MR data used for the reconstruction of an MR image were data acquired around that time point assigned to the MR image. It is also possible according to the invention to take all of the acquired MR data into account for the reconstruction of an MR image or each MR image. In this embodiment, effectively each MR image is accordingly dependent on each set of MR data or each part of the MR data.

A magnetic resonance system for generating MR images of a predetermined volume segment in an examination subject is also provided within the scope of the present invention. Such a magnetic resonance system has a basic field magnet, a gradient field system, at least one RF antenna, and a control device that operates the gradient field system and the at least one RF antenna, for receiving measured signals picked up by the RF antenna or antennas and for evaluating the measured signals, and for generating the MR images. The magnetic resonance system is designed to be operated to acquire MR data within the volume segment with the RF antenna and the gradient field system, using the same measurement configuration. The control device is furthermore configured to reconstruct multiple MR images from the MR data. In this case each of these MR images is assigned to an individual time point at which the MR image maps (represents) at least a specific part of the volume segment. The spatial resolution during the acquisition of the MR data is constant, and the temporal distance between each two time points succeeding one another in time is not constant.

The advantages of the magnetic resonance system according to the invention substantially correspond to the advantages of the method according to the invention, as explained in detail.

The present invention also encompasses a non-transitory, computer-readable data storage medium that can be loaded into a memory of a programmable controller or a computing unit of a magnetic resonance system. The storage medium is encoded with programming instructions (code) that cause all or various of the described embodiments of the method according to the invention to be performed by the computer serving as the controller or control device of the magnetic resonance system. For this, the programming instructions may possibly require other program means, e.g. libraries and auxiliary functions, in order to realize the corresponding embodiments of the methods. The code can be a source code (e.g. C++) that still needs to be compiled (assembled) and linked or that only needs to be interpreted, or can be an executable software code that only needs to be loaded into the corresponding computing unit or control device in order to be executed.

The electronically readable data medium can be, for example, a DVD, a magnetic tape or a USB stick on which electronically readable control information, in particular software, is stored.

By the execution of the present invention, it is possible to reconstruct MR images of a three-dimensional volume segment or else only of a two-dimensional volume segment (of a slice). The present invention may be employed for spin-echo-based and for gradient-echo-based methods. K-space can be sampled using a Cartesian scheme or radially. In addition, known prior art methods can be used in order for example to reduce the so-called flickering between reconstructed MR images succeeding one another in time.

The present invention enables MR data to be acquired at a high temporal resolution during, for example, free breathing by the subject, without the expense of a very protracted reconstruction of the MR images. Because, according to the invention, the MR images are reconstructed at a high temporal resolution only in phases of interest (e.g. only in a specific breathing phase), whereas in the other breathing phases the MR images are reconstructed only at a very low temporal resolution, the computing time for the reconstruction as a whole can be kept short.

### BRIEF DESCRIPTION OF THE DRAWINGS

FIG. 1 shows a magnetic resonance system according to the invention in a schematic representation.

FIGS. 2a-2d show assignments of MR data to MR images that are to be reconstructed.

FIG. 3 is a flowchart of an embodiment of the method according to the invention.

FIG. 4 shows a measured value curve at the time of injection of a contrast agent.

### DESCRIPTION OF THE PREFERRED EMBODIMENTS

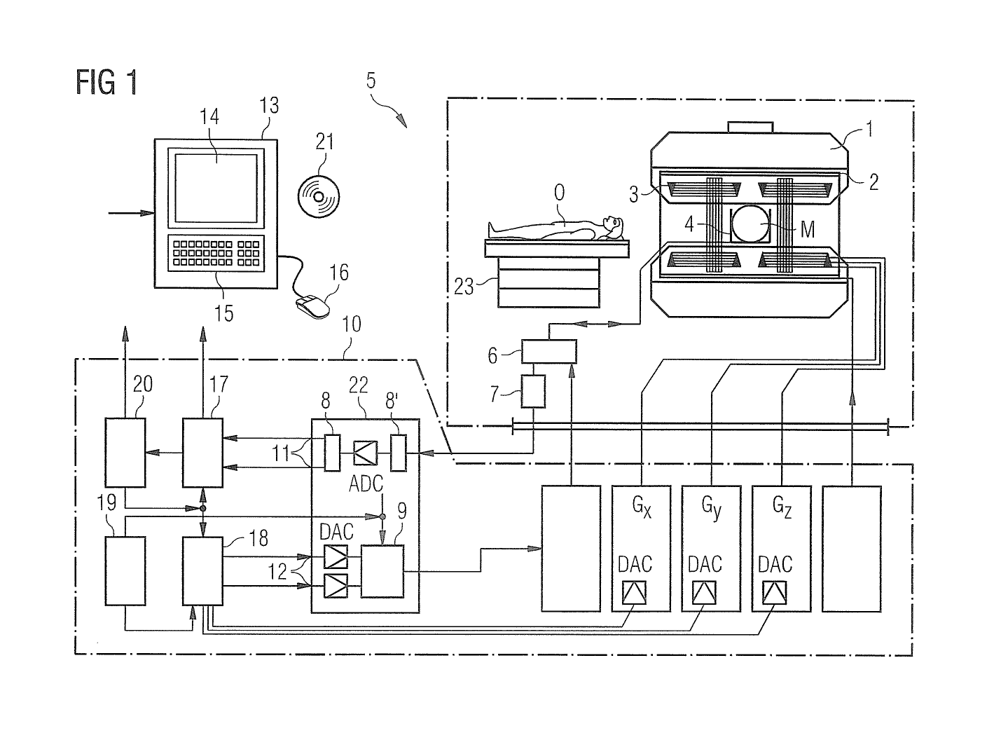

FIG. 1 shows a schematic illustration of a magnetic resonance system 5 (of a magnetic resonance imaging or nuclear spin tomography apparatus). In this case a basic field magnet 1 generates a temporally constant, strong magnetic field for the polarization or alignment of the nuclear spins in an examination region of a subject O, such as e.g. a part that is to be examined of a human body which, lying supine on a table 23, is continuously introduced into the magnetic resonance system 5. The high homogeneity of the basic magnetic field required for the nuclear magnetic resonance measurement is defined in a typically spherical measurement volume M through which the parts of the human body that are to be examined are e.g. continuously introduced. In order to support the homogeneity requirements and in particular to eliminate time-invariable influences, so-called shim plates made of ferromagnetic material are installed at suitable points. Time-variable influences are eliminated by the operation of shim coils 2.

A cylindrical gradient field system or gradient field system 3 composed of three sub-windings is inserted into the basic field magnet 1. Each sub-winding is supplied with electrical power by an amplifier in order to generate a linear (also time-variable) gradient field in the respective direction of the Cartesian coordinate system. In this case the first sub-winding of the gradient field system 3 generates a gradient Gₓ in the x-direction, the second sub-winding a gradient G_{y} in the y-direction, and the third sub-winding a gradient G_{z}, in the z-direction. The amplifier includes a digital-to-analog converter that is operated by a sequence controller 18 to assure the correctly timed generation of gradient pulses.

Disposed within the gradient field system 3 are one or more radiofrequency antennas 4 that convert the radiofrequency pulses emitted by a radiofrequency power amplifier into an alternating magnetic field in order to excite the nuclei and deflect (flip) the nuclear spins of the examination subject O or the region of the subject O that is to be examined, from the alignment with the basic magnetic field. Each radiofrequency antenna 4 is composed of one or more RF transmit coils and one or more RF receive coils in the form of an annular, preferably linear or matrix-shaped array of component coils. The RF receive coils of the respective radiofrequency antenna 4 also convert the alternating field emanating from the precessing nuclear spins, i.e. usually the nuclear spin echo signals produced by a pulse sequence composed of one or more radiofrequency pulses and one or more gradient pulses, into a voltage (measured signal or measured value) which is supplied via an amplifier 7 to a radiofrequency receive channel 8 of a radiofrequency system 22. The radiofrequency system 22, which is part of a control device 10 of the magnetic resonance system 5, additionally includes a transmit channel 9 in which the radiofrequency pulses for exciting the magnetic nuclear resonance are generated. Based on a pulse sequence predefined by the system computer 20 in the sequence controller 18, the respective radiofrequency pulses are represented digitally as a sequence of complex numbers. This number sequence is supplied in the form of a real part and an imaginary part via respective inputs 12 to a digital-to-analog converter in the radiofrequency system 22, and from this converter to a transmit channel 9. In the transmit channel 9, the pulse sequences are modulated onto a radiofrequency carrier signal having a fundamental frequency that corresponds to the resonant frequency of the nuclear spins in the measurement volume.

The switchover from transmit to receive mode is accomplished via a transmit-receive duplexer 6. The RF transmit coils of the radiofrequency antenna(s) 4 beam the radiofrequency pulses for exciting the nuclear spins into the measurement volume M and resulting echo signals are sampled via the RF receive coil(s). The correspondingly obtained nuclear magnetic resonance signals are demodulated in the receive channel 8′ (first demodulator) of the radiofrequency system 22 in a phase-sensitive manner onto an intermediate frequency, digitized in the analog-to-digital converter (ADC), and emitted via the output 11. The signal is further demodulated onto the frequency 0. The demodulation onto the frequency 0 and the separation into real and imaginary parts takes place after the digitization in the digital domain in a second demodulator 8. An MR image can be reconstructed by an image computer 17 from the measurement data obtained in that way via an output 11. The management of the measurement data, the image data and the control programs is handled via the system computer 20. Based on a specification by means of control programs, the sequence controller 18 monitors and controls the generation of the pulse sequences desired in each case and the corresponding sampling of k-space. In particular the sequence controller 18 controls the correctly timed switching of the gradients, the transmitting of the radiofrequency pulse at the defined phase amplitude and the reception of the nuclear magnetic resonance signals. The time base for the radiofrequency system 22 and the sequence controller 18 is provided by a synthesizer 19. Appropriate control programs for generating an MR image, which are stored e.g. on a DVD 21, are selected and the generated MR image is displayed at a terminal 13, which has a keyboard 15, a mouse 16 and a monitor screen 14.

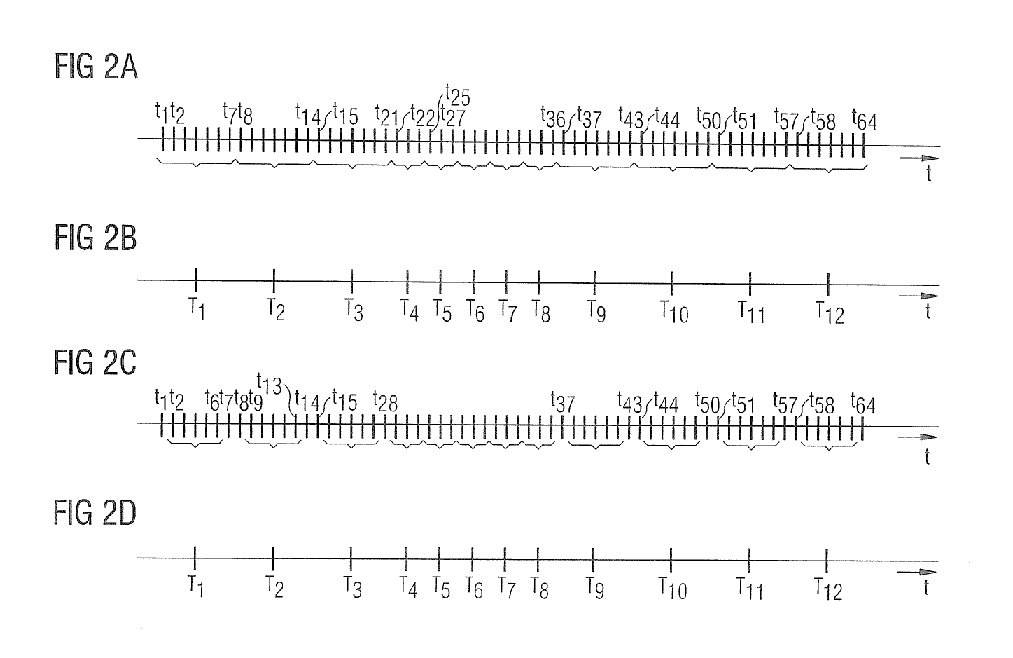

FIG. 2a shows sixty-four time points t₁ to t₆₄ at which MR data are acquired. It should be pointed out that the MR data are actually acquired not at a time point, but during a time interval. To simplify the discussion it is assumed that this time interval starts in each case before the respective time point and ends after the respective time point, and can therefore be represented by the respective time point.

FIG. 2b shows twelve time points T₁ to T₁₂ which are each assigned to an MR image that is to be reconstructed. It can be seen that the temporal resolution of the MR images to be reconstructed is lower at the start (T₁ to T₃) and at the end (T₁₀ to T₁₂) than in the middle (T₄ to T₈). In other words, the temporal distance between two succeeding MR images that are to be reconstructed is greater at the start (T₁ to T₃) and at the end (T₁₀ to T₁₂) than in the middle (T₄ to T₈).

According to the embodiment illustrated by FIGS. 2a and 2b, the MR data acquired at time points t₁ to t₇ are used for reconstructing the MR image assigned to time point T₁, whereas only the MR data acquired at time points t₂₅ to t₂₇ are used for example for reconstructing the MR image assigned to time point T₅. It can be seen, therefore, that more MR data are used in each case at the start (T₁ to T₃) and at the end (T₁₀ to T₁₂) for reconstructing the MR images than in the middle (T₄ to T₈) if it is assumed that the volume of MR data acquired at a time point t₁ to t₆₄ are constant.

A further embodiment according to the invention is illustrated in FIGS. 2c and 2d. FIG. 2c once again shows sixty-four time points t₁ to t₆₄ at which MR data is (can be) acquired, and FIG. 2d once again shows the same twelve time points T₁ to T₁₂ which correspond to time points T₁ to T₁₂ in FIG. 2b and which are each assigned to an MR image that is to be reconstructed.

In contrast to the embodiment illustrated in FIGS. 2a and 2b, however, only MR data acquired at five time points in each case are now used for reconstructing the MR images assigned to time points T₁ to T₃ and T₉ to T₁₂. In this case MR data that are not used for reconstructing one of the MR images may be acquired, but not used, or else not acquired at all in the first place.

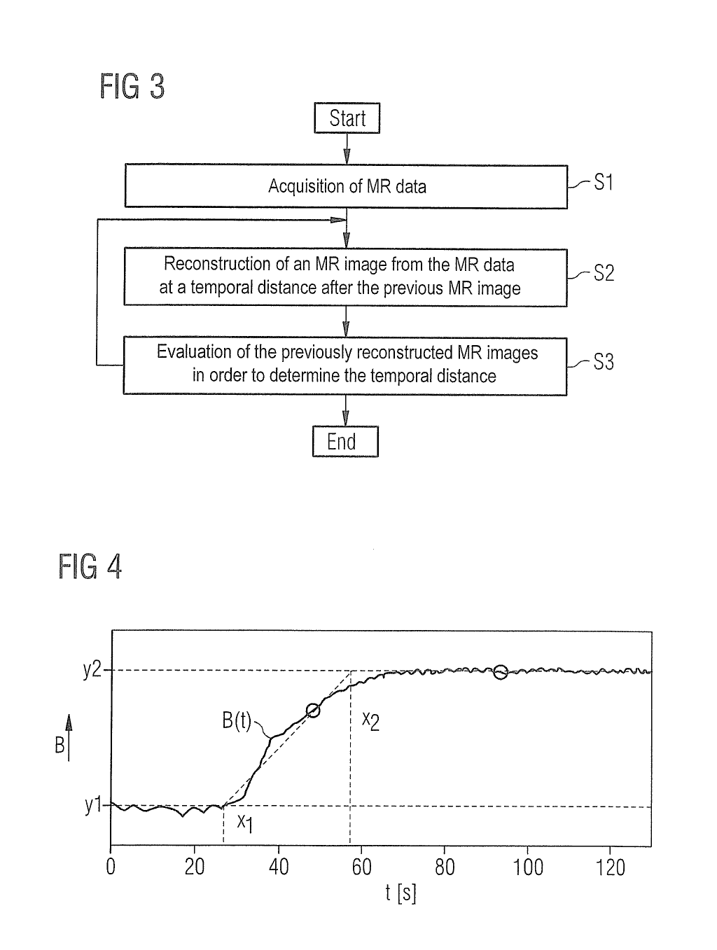

FIG. 3 shows a flowchart of a method according to the invention.

The MR data is acquired in the first step S1. In the loop consisting of the following steps S2 and S3, an MR image is reconstructed each time in step S2 from the respective MR data. In this case the temporal distance between the time point assigned to the MR image that is currently to be reconstructed and the directly preceding time point corresponding to the MR image reconstructed directly beforehand is set as a function of information ascertained from step S3. In step S3, the respective MR images reconstructed thus far are evaluated in order for example to establish the development of a contrast agent concentration so as to determine the temporal distance as a function thereof.

FIG. 4 shows a measured value curve B(t) over time by means of which the development of a contrast agent concentration in the examination subject O is mapped. The typical time curve B(t) of such an accumulation of a contrast agent can be replicated by a first linear section running parallel to the time axis, followed by a second linear section having a constant incline, and a subsequent third linear section which in turn runs parallel to the time axis. In particular time point x₁, at which the first linear section ends and the second linear section begins, and time point x₂, at which the second linear section ends and the third linear section begins, are of interest in this case. Whereas time point x₁ corresponds to the time point at which the contrast agent previously injected into the examination subject diffuses in the observed volume segment, and therefore the concentration of the contrast agent increases, time point x₂ corresponds to the time point at which the concentration of the contrast agent in the observed volume segment has reached the maximum value and the so-called wash-out phase begins.

By determining x1 and x2 it is possible to determine the time range of the contrast agent uptake and select a higher temporal resolution in said time range than at earlier or later time points. If, for example, MR data is acquired in the time period t=0 to t=120, MR images in the time interval x₁ to x₂, i.e. during the increase in the contrast agent concentration in the observed volume segment, are of interest in particular. The MR images to be reconstructed should therefore have a higher temporal resolution during said time interval x₁ to x₂ than, for example, at times before x₁ or at times after x₂. In other words, the time curve of the contrast agent concentration in the observed volume segment in the time interval x₁ to x₂ could be visualized by means of reconstructed MR images at intervals of 5 s, whereas reconstructed MR images are present only every 30 s for times before x₁ or after x₂.

While the embodiment illustrated by means of FIG. 3 makes use of previously reconstructed MR images in order to ascertain the information (for example time points x₁ and x₂) as a function of which the temporal resolution of the MR images to be reconstructed is determined, this information can also be ascertained on the basis of the MR data itself, as is described hereinbelow.

The volume segment to be observed may be sampled using the so-called stack-of-stars method. In this process, the volume segment is sampled one slice at a time, with each slice being sampled by sampling the k-space corresponding to the respective slice on the basis of spokes (referred to as stars) running radially through the center. In this case the absolute amount of the value for the k-space point directly before the center, the absolute amount of the value for the k-space point in the center, and the absolute amount of the value for the k-space point directly after the center are determined for each radial spoke, and the average value is formed from said three amounts. This average value then corresponds to the absolute amount B(t), where t corresponds to the time point at which the corresponding radial spoke is acquired.

The time points x₁, x₂ of interest can be determined as a function of the absolute amounts Bᵢ determined at the respective time point i by the following equation (1) by determining a minimum for the cost function f(x₁, x₂).

$$f ( {x}_{1} , {x}_{2} ) = {\sum }_{i = 1}^{{x}_{1}} {( {B}_{i} - {y}_{1} )}^{2} + {\sum }_{i = {x}_{1} + 1}^{{x}_{2} - 1} {( {B}_{i} - \frac{{y}_{2} - {y}_{1}}{{x}_{2} - {x}_{1}} \times ( i - {x}_{1} ) - {y}_{1} )}^{2} + {\sum }_{i = {x}_{2}}^{N} {( {B}_{i} - {y}_{2} )}^{2} \qquad ( 1 )$$

where y₁ can correspond for example to the average value of the absolute amounts Bᵢ determined for the first time points, and y₂ can correspond for example to the average value of the absolute amounts Bᵢ determined for the last time points. N corresponds to the number of all-time points (more than 120 in FIG. 4).

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for generating magnetic resonance (MR) images, comprising:
operating an MR scanner while an examination subject is situated therein to acquire MR data from a predetermined volume segment within the examination subject, while maintaining said MR scanner with a same measurement configuration during acquisition of all of said MR data and, by said same measurement configuration, causing of all said MR data to be acquired with a spatial resolution that is constant during the acquisition of the MR data;
providing the acquired MR data to a computer and, in said computer, reconstructing a plurality of MR images from the MR data, and assigning each of said MR images a respective time point at which the respective MR image represents at least a part of said volume segment;
in said computer, making a temporal spacing, between each two successive time points, not constant; and
from said computer, making said MR images available in electronic form as a data file.

2. A method as claimed in claim 1 comprising setting said measurement configuration of said MR scanner by a calibration of said MR scanner that sets at least one of a transmit power of a radio frequency (RF) transmitter of said MR scanner, a reception sensitivity of an RF receiver of said MR scanner, and an excitation frequency of RF energy emitted by said RF transmitter.

3. A method as claimed in claim 1 comprising, in said computer, determining respective temporal spacings between each two successive time points dependent on information that describes a change that occurs within said volume segment during the acquisition of said MR data.

4. A method as claimed in claim 1 comprising, in said computer, determining said information from the acquired MR data, before any MR images are reconstructed from said MR data.

5. A method as claimed in claim 4 comprising entering the acquired MR data into an electronic memory organized as k-space, said computer having access to said memory, and, in said computer, determining said information from data entered into a slice in k-space that proceeds through a center of k-space, or from data surrounding said center of k-space.

6. A method as claimed in claim 4 comprising administering a contrast agent to the examination subject preceding the acquisition of said MR data and, in said computer, determining said information to comprise a first informational point in time at which an increase in a concentration of said contrast agent in the volume segment can be determined from the acquired MR data, and a second informational point in time at which an end of said increase can be deduced from the acquired MR data.

7. A method as claimed in claim 6 comprising setting the respective temporal spacings between each two successive time points so as to satisfy at least one of:
the temporal spacing between each two successive time points that occur before said first informational point in time is greater than a temporal spacing between each two successive time points that occur after said first informational point in time and before said second informational point in time; and
the temporal spacing between each two successive time points that occur after said second informational point in time is greater than the temporal spacing between each two successive time points that occur after said first informational point in time and before said second informational point in time.

8. A method as claimed in claim 3 comprising deriving said information in said computer from the reconstructed MR images.

9. A method as claimed in claim 3 comprising:
administering a contrast agent to the examination subject prior to acquiring said MR data; and
in said computer, using, as said information, an injection point in time at which said contrast agent is administered to the examination subject.

10. A method as claimed in claim 1 comprising reconstructing the respective MR images from only a portion of the acquired MR data that were acquired during a first predetermined time period before and a second predetermined time period after the time point assigned to the respective MR image.

11. A method as claimed in claim 1 comprising using all of the acquired MR data for the reconstruction of said MR images.

12. A method as claimed in claim 1 comprising, in said computer, if a temporal spacing between a first of said time points and a second of said time points that immediately follows said first of said time points is greater than a predetermined time threshold value, not using a portion of said MR data that was acquired between said first of said time points and said second of said time points for reconstruction of any of said MR images.

13. A method as claimed in claim 1 comprising, in said computer, if a temporal spacing between a first of said time points and a second of said time points that immediately follows said first of said time points is greater than a predetermined time threshold value, not acquiring MR data between said first of said time points and said second of said time points.

14. A method as claimed in claim 1 comprising operating said MR scanner to acquire more MR data for reconstructing at least one of said MR images as said temporal distance becomes larger between the time point assigned to a respective MR image and a time point assigned to a next MR image that is to be reconstructed.

15. A method as claimed in claim 1 comprising operating said MR scanner to acquire said MR data with a constant temporal resolution.

16. A method as claimed in claim 1 comprising using all of the acquired MR data for reconstructing each of said MR images.

17. A magnetic resonance (MR) apparatus comprising:
an MR scanner comprising a basic field magnet, a gradient field system, at least one radio frequency (RF) transmitter and at least one RF receiver;
a control unit configured to operate said MR scanner, while an examination subject is situated therein, to acquire MR data from a predetermined volume segment of the examination subject with said basic field magnet, said gradient field system, said RF transmitter and said RF receiver being maintained in a same measurement configuration during acquisition of all of said MR data, said same measurement configuration giving said MR data a constant spatial resolution during acquisition of all of said MR data;
an image reconstruction computer provided with said MR data, said image reconstruction computer being configured to reconstruct a plurality of MR images from the acquired MR data, and to assign each of said MR images a respective time point at which the respective MR image represents at least a portion of said volume segment;
said image reconstruction computer being configured to set a temporal spacing between each two successive time points that is not constant; and
said image reconstruction computer being configured to make the reconstructed MR images available at an output thereof in electronic form as a data file.

18. A non-transitory, computer-readable data storage medium encoded with programming instructions, said storage medium being loaded into a control and processing computer system of a magnetic resonance (MR) apparatus, said MR apparatus comprising an MR scanner, and said programming instructions causing said control and processing computer system to:
operate said MR scanner while an examination subject is situated therein to acquire MR data from a predetermined volume segment within the examination subject, while maintaining said MR scanner with a same measurement configuration during acquisition of all of said MR data and, by said same measurement configuration, causing of all said MR data to be acquired with a spatial resolution that is constant during the acquisition of the MR data;
reconstruct a plurality of MR images from the MR data, and assign each of said MR images a respective time point at which the respective MR image represents at least a part of said volume segment;
make a temporal spacing, between each two successive time points, not constant; and
make said MR images available in electronic form as a data file.
